(19) United States
(12) Patent Application Publication
(10) Pub. No.: US 2006/0019406 A1
(43) Pub. Date: Jan. 26, 2006

(54) Lateral flow device for the detection of large pathogens

(76) Inventors: Ning Wei, Roswell, GA (US); Shu-Ping Yang, Alpharetta, GA (US)

Correspondence Address: KIMBERLY-CLARK WORLDWIDE, INC., 401 NORTH LAKE STREET, NEENAH, WI, 54956, US

(21) Appl. No.: 10/898,059

(22) Filed: Jul. 23, 2004

(51) Int. Cl. G01N 33/558

(52) U.S. Cl. 436/514

(57) ABSTRACT

There is provided a lateral flow assay device for detecting the presence or quantity of an analyte residing in a test sample where the lateral flow assay device has a porous membrane in communication with a conjugate pad and a wicking pad. The porous membrane has a detection zone where a test sample is applied and which has an immobilized first capture reagent configured to bind to at least a portion of the analyte and analyte-conjugate complexes to generate a detection signal. The control zone is located downstream from the detection zone on the porous membrane and has a second capture reagent immobilized within the control zone. The conjugate pad is located upstream from the detection zone, and has detection probes with specific binding members for the analyte. A buffer release zone is located upstream of the conjugate zone and provides for buffer addition to the device, the buffer serving to move the detection probes to the detection and control zones.

### BACKGROUND OF THE INVENTION

The diagnosis of large pathogens is currently performed by examining samples under a microscope or by culturing a specimen. Microscopic evaluation requires a trained specialist and an instrument while culturing specimens generally requires a time of more than 24 hours to obtain results.

Flow through assays have thus far proven of limited use in detection of large pathogens because of the size of the pathogen. For example, various analytical procedures and devices are commonly employed in lateral flow assays to determine the presence and/or concentration of smaller analytes that may be present in a test sample. Immunoassays, for example, utilize mechanisms of the immune systems, where antibodies are produced in response to the presence of antigens that are pathogenic or foreign to the organisms. These antibodies and antigens, i.e., immunoreactants, are capable of binding with one another, thereby causing a highly specific reaction mechanism that may be used to determine the presence or concentration of that particular antigen in a biological sample. These assays require the movement of the analyte through the device, thus hindering their usefulness with larger, lower mobility, pathogens.

There are several well-known immunoassay methods that use immunoreactants labeled with a detectable component so that the analyte may be detected analytically. For example, “sandwich-type” assays typically involve mixing the test sample with detectable probes, such as dyed latex or a radioisotope, which are conjugated with a specific binding member for the analyte. The conjugated probes form complexes with the analyte. These complexes then reach a zone of immobilized antibodies where binding occurs between the antibodies and the analyte to form ternary “sandwich complexes.” The sandwich complexes are localized at the zone for detection of the analyte. This technique may be used to obtain quantitative or semi-quantitative results.

An alternative technique is the “competitive-type” assay. In a “competitive-type” assay, the label is typically a labeled analyte or analyte-analogue that competes for binding of an antibody with any unlabeled analyte present in the sample. Competitive assays are typically used for detection of analytes such as haptens, each hapten being monovalent and capable of binding only one antibody molecule.

Despite the benefits achieved from these devices, many conventional lateral flow assays encounter significant inaccuracies when exposed to relatively high analyte concentrations and when attempting to detect very large pathogens that are difficult to cause to flow. When the analyte is present at high concentrations, for example, a substantial portion of the analyte in the test sample may not form complexes with the conjugated probes. Thus, upon reaching the detection zone, the uncomplexed analyte competes with the complexed analyte for binding sites. Because the uncomplexed analyte is not labeled with a probe, it cannot be detected. Consequently, if a significant number of the binding sites become occupied by the uncomplexed analyte, the assay may exhibit a “false negative.” This problem is commonly referred to as the “hook effect.” In the case of large pathogens, like, for example, Candida albican, it is likely that the complex will not properly flow to the detection zone on the membrane because of the size of the complex formed.

A need still exists, however, for an improved technique of reducing the “hook effect” and of detecting large pathogens that are difficult to cause to flow through a lateral flow device.

### SUMMARY OF THE INVENTION

In accordance with one embodiment of the present invention, an assay device for detecting the presence or quantity of a large analyte residing in a test sample is disclosed. The assay device comprises a conjugate pad that is in liquid communication with a porous membrane that is also in communication with a wicking pad.

The porous membrane may be made from any of a variety of materials through which the detection probes are capable of passing like, for example, nitrocellulose. The porous membrane has a detection zone where a test sample is contacted, deposited or applied and within which is immobilized a first capture reagent. The first capture reagent is configured to bind to at least a portion of the analyte and analyte-conjugate complexes to generate a detection signal. The first capture reagent may be selected from the group consisting of antigens, haptens, protein A or G, neutravidin, avidin, streptavidin, captavidin, primary or secondary antibodies, and complexes thereof. The first capture reagent may, for example, bind to complexes formed between the analyte and the conjugated detection probes.

The control zone is located on the porous membrane downstream from the detection zone. A second capture reagent is immobilized within the control zone that is configured to bind to the conjugate, conjugate-analyte complex or pure probes, to indicate the assay is performing properly. In one embodiment, the second capture reagent is selected from the group consisting of antigens, haptens, protein A or G, neutravidin, avidin, streptavidin, captavidin, primary or secondary antibodies, and complexes thereof.

The conjugate pad contains detection probes that signal the presence of the analyte. The conjugate pad may also include other, different probe populations, including probes for indication at the control zone. If desired, the detection probes may comprise a substance selected from the group consisting of chromogens, catalysts, luminescent compounds (e.g., fluorescent, phosphorescent, etc.), radioactive compounds, visual labels, liposomes, and combinations thereof. The specific binding member may be selected from the group consisting of antigens, haptens, aptamers, primary or secondary antibodies, biotin, and combinations thereof.

In liquid communication with the end of the conjugate pad away from the membrane there is a buffer release zone. After the sample has been deposited on the detection zone, a buffer is released from upstream of the conjugate pad in the buffer release zone. The buffer washes probes from the conjugate pad toward the detection zone where the detection probes will be captured on the detection zone by the analyte, if present, and yield a positive result. If the sample contains no analyte, the detection line will be negative. The buffer, still containing some probes (which may include probes different from the detection probes) continues to the control zone where a reagent captures conjugate, conjugate-analyte complex or pure probes to indicate the assay is functioning properly.

The wicking pad is in liquid communication with the membrane and provides a driving force for liquid movement due to the capillarity of the pad.

In accordance with another embodiment of the present invention, a method for detecting the presence or quantity of an analyte residing in a test sample is disclosed. The method includes the steps of
i) providing a lateral flow assay device having a porous membrane in liquid communication with a conjugate pad and a wicking pad, the conjugate pad having detection probes conjugated with a specific binding member for the analyte, the porous membrane defining a detection zone in which a first capture reagent is immobilized and a control zone within which a second capture reagent is immobilized, wherein the control zone is located downstream from the detection zone, the conjugate pad is located upstream of the porous membrane and the buffer release zone is upstream of the conjugate pad;
ii) contacting the test sample containing the analyte with the detection zone;
iii) releasing a buffer at the buffer release zone so that the buffer will carry the detection probes to the detection and control zones;
iv) detecting the detection signal.

Other features and aspects of the present invention are discussed in greater detail below.

### BRIEF DESCRIPTION OF THE DRAWINGS

FIG. 1 is a perspective view of one embodiment of a lateral flow assay device of the present invention.

### DETAILED DESCRIPTION

As used herein, the term “analyte” generally refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include ferritin; creatinine kinase MB (CK-MB); digoxin; phenyloin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyhene. Other potential analytes may be described in U.S. Pat. No. 6,436,651.

As used herein, the term “test sample” generally refers to a material suspected of containing the analyte. The test sample may, for instance, include materials obtained directly from a source, as well as materials pretreated using techniques, such as, but not limited to, filtration, precipitation, dilution, distillation, mixing, concentration, inactivation of interfering components, the addition of reagents, and so forth. The test sample may be derived from a biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, amniotic fluid or the like. Besides physiological fluids, other liquid samples may be used, such as water, food products, and so forth. In addition, a solid material suspected of containing the analyte may also be used as the test sample.

In general, the present invention is directed to a lateral flow assay device for detecting the presence or quantity of an analyte residing in a test sample. Known assays require that the pathogens move from a point of deposition to a point where they may be detected. Rather than move the pathogens through an area containing detection probes and then to a detection zone, however, the instant invention moves the probes, initially located on a conjugate pad, to the pathogen located in a detection zone having a capture reagent. The inventors have discovered that allowing the detection probes to move to the sample, instead of the general practice which is the reverse, enables the detection of large analytes over extended concentration ranges in a simple, efficient, and cost-effective manner. It also is suitable for the detection of smaller pathogens, particularly at lower concentrations, and virtually eliminates the “hook effect” caused by an excess of uncomplexed analyte.

The device utilizes a porous membrane having a detection zone and a control zone. The detection and control zones have immobilized capture reagents. The device further uses a buffer release zone on the upstream end of the device and a conjugate pad located between the buffer release zone and the porous membrane. A wicking pad is in liquid communication with the opposite end of the porous membrane on the downstream end of the device. In use, the sample is applied in the detection zone and after a period of time, the buffer is released. The buffer washes detection and optionally other types of probes, from the conjugate pad through the detection zone, resulting in an indication of the presence of pathogens.

The preferred pathogens for analysis in the present invention are those that are relatively large, i.e.; between about 0.03 and 30 microns in size. Large pathogens are difficult to detect using currently known lateral flow devices because their size makes them difficult to move.

Examples of suitable pathogens that may be detected using the invention include, but are not limited to bacteria such as Salmonella species, Neisseria meningitides groups, Streptococcus pneumoniae, yeasts such as Candida albicans, Candida tropicalis, fungi such as aspergillua, viruses such as haemophilus influenza, HIV, and protozoa such as Trichomonas and Plasmodium.

While larger pathogens are preferred, the assay of the present invention is also suitable for smaller pathogens (analytes), e.g. less than 0.3 microns in size. When the small analyte is present in a low concentration it may be so dispersed or diluted and too insufficient in quantity to be noted at the detection zone of conventional lateral flow devices. Depositing the test sample at the detection zone increases the likelihood of detection for low concentration, small pathogens. When the small analyte is present in a high concentration, the “hook effect” common to conventional assays may be avoided, as discussed further below. Additionally, small pathogens do not move well through the membrane if the porous membrane is one with relatively large pores. If this is the case, false negative results are again possible due to the lack of mobility of the pathogen to the detection zone. The instant invention overcomes these failures to detect small pathogens by depositing the test sample directly onto the detection zone.

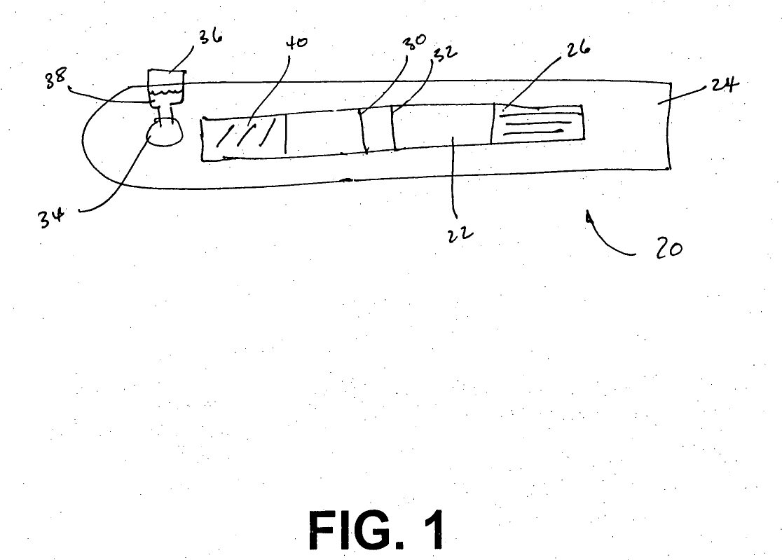

Referring to FIG. 1, one embodiment of a lateral flow assay device 20 that may be formed according to the present invention will now be described in more detail. It should be noted that the term “lateral flow” is meant to be descriptive and not limiting, as the device could be configured in other ways with the same effect. Radial or vertical flow devices can easily be envisioned, for example, employing the same principle as the instant invention, without departure from the spirit of the invention. As shown, the device 20 contains a porous membrane 22 optionally supported by a rigid material 24. The porous membrane 22 has a detection zone (or line) 30. The porous membrane 22 also has a control zone (or line) 32.

In general, the porous membrane 22 may be made from any of a variety of materials through which the detection probes are capable of passing. For example, the materials used to form the porous membrane 22 may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and the like. In one particular embodiment, the porous membrane 22 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The device 20 may also contain a wicking pad 26. The wicking pad 26 generally receives fluid that has migrated through the entire porous membrane 22. As is well known in the art, the wicking pad 26 may assist in promoting capillary action and fluid flow through the membrane 22.

The device 20 has a buffer release zone 34. In one embodiment the buffer release zone 34 has a buffer reservoir 36 within which may be stored the buffer 38. Buffer 38 may alternatively be supplied by a separate reservoir. The buffer 28 may be any liquid that will carry away the detection probes used in the invention. Examples of suitable buffers include phosphate buffered saline (PBS) solution (pH of 7.2), tris-buffered saline (TBS) solution (pH of 8.2) or 2-(N-morpholino) ethane sulfonic acid (MES) (pH of 5.3).

A conjugate pad 40 is in liquid communication with the buffer release zone 34 and is located between the buffer release zone 34 and the porous membrane 22 so that as the buffer 38 moves from the buffer release zone 34 it will traverse the conjugate pad 40 and carry probes to the detection zone 30 and the control zone 32 on the porous membrane 22. The conjugate pad 40 is formed from a material through which the buffer is capable of passing. The conjugate pad 40 may be formed from glass fibers, for example. Although only one conjugate pad 40 is shown, it should be understood that other conjugate pads may also be used in the present invention.

To initiate the detection of an analyte within the test sample, a user may directly apply, contact or deposit the test sample to the detection zone 30 portion of the porous membrane 22. In the illustrated embodiment, the test sample is placed in the detection zone 30. Once the sample has contacted the detection zone 30, buffer 38 is released into the buffer release zone 34. The buffer 38 may be applied by means of an integral reservoir, or by a separate source such as by pipette or any other effective means known to those skilled in the art. The buffer 38 travels through the conjugate pad 40 that is in liquid communication with the porous membrane 22, to the detection zone 30 and the control zone 32.

A predetermined amount of at least one type of detection probes are applied on the conjugate pad in order to facilitate accurate detection of the presence or absence of an analyte within the test sample. Any substance generally capable of generating a signal that is detectable visually or by an instrumental device may be used as detection probes. Various suitable substances may include chromogens; catalysts; luminescent compounds (e.g., fluorescent, phosphorescent, etc.); radioactive compounds; visual labels, including colloidal metallic (e.g., gold) and non-metallic particles, dye particles, enzymes or substrates, or organic polymer latex particles; liposomes or other vesicles containing signal producing substances; and so forth. Some enzymes suitable for use as detection probes are disclosed in U.S. Pat. No. 4,275,149. One example of an enzyme/substrate system is the enzyme alkaline phosphatase and the substrate nitro blue tetrazolium-5-bromo-4-chloro-3-indolyl phosphate, or derivative or analog thereof, or the substrate 4-methylumbelliferyl-phosphate. Other suitable detection probes may be described in U.S. Pat. Nos. 5,670,381 and 5,252,459.

In some embodiments, the detection probes may contain a fluorescent compound that produces a detectable signal. The fluorescent compound may be a fluorescent molecule, polymer, dendrimer, particle, and so forth. Some examples of suitable fluorescent molecules, for instance, include, but are not limited to, fluorescein, europium chelates, phycobiliprotein, rhodamine and their derivatives and analogs.

The detection probes, such as described above, may be used alone or in conjunction with a microparticle (sometimes referred to as “beads” or “microbeads”). For instance, naturally occurring microparticles, such as nuclei, mycoplasma, plasmids, plastids, mammalian cells (e.g., erythrocyte ghosts), unicellular microorganisms (e.g., bacteria), polysaccharides (e.g., agarose), and so forth, may be used. Further, synthetic microparticles may also be utilized. For example, in one embodiment, latex microparticles that are labeled with a fluorescent or colored dye are utilized. Although any latex microparticle may be used in the present invention, the latex microparticles are typically formed from polystyrene, butadiene styrenes, styreneacrylic-vinyl terpolymer, polymethylmethacrylate, polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. Other suitable microparticles may be described in U.S. Pat. Nos. 5,670,381 and 5,252,459. Commercially available examples of suitable fluorescent particles include fluorescent carboxylated microspheres sold by Molecular Probes, Inc. under the trade names “FluoSphere” (Red 580/605) and “TransfluoSphere” (543/620), as well as “Texas Red” and 5- and 6-carboxytetramethylrhodamine, which are also sold by Molecular Probes, Inc. In addition, commercially available examples of suitable colored, latex microparticles include carboxylated latex beads sold by Bang's Laboratory, Inc.

When utilized, the shape of the particles may generally vary. In one particular embodiment, for instance, the particles are spherical in shape. However, it should be understood that other shapes are also contemplated by the present invention, such as plates, rods, discs, bars, tubes, irregular shapes, etc. In addition, the size of the particles may also vary. For instance, the average size (e.g., diameter) of the particles may range from about 0.1 nanometers to about 1,000 microns, in some embodiments, from about 0.1 nanometers to about 100 microns, and in some embodiments, from about 1 nanometer to about 10 microns. For instance, “micron-scale” particles are often desired. When utilized, such “micron-scale” particles may have an average size of from about 1 micron to about 1,000 microns, in some embodiments from about 1 micron to about 100 microns, and in some embodiments, from about 1 micron to about 10 microns. Likewise, “nano-scale” particles may also be utilized. Such “nano-scale” particles may have an average size of from about 0.1 to about 10 nanometers, in some embodiments from about 0.1 to about 5 nanometers, and in some embodiments, from about 1 to about 5 nanometers.

In some instances, it is desired to modify the detection probes in some manner so that they are more readily able to bind to the analyte. In such instances, the detection probes may be modified with certain specific binding members that are adhered thereto to form conjugated probes. Specific binding members generally refer to a member of a specific binding pair, i.e., two different molecules where one of the molecules chemically and/or physically binds to the second molecule. For instance, immunoreactive specific binding members may include antigens, haptens, aptamers, antibodies (primary or secondary), and complexes thereof, including those formed by recombinant DNA methods or peptide synthesis. An antibody may be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art. Other common specific binding pairs include but are not limited to, biotin and avidin (or derivatives thereof), biotin and streptavidin, carbohydrates and lectins, complementary nucleotide sequences (including probe and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and so forth. Furthermore, specific binding pairs may include members that are analogs of the original specific binding member. For example, a derivative or fragment of the analyte, i.e., an analyte-analog, may be used so long as it has at least one epitope in common with the analyte.

The specific binding members may generally be attached to the detection probes using any of a variety of well-known techniques. For instance, covalent attachment of the specific binding members to the detection probes (e.g., particles) may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the detection probe may contain a relatively high surface concentration of polar groups. In addition, although detection probes are often functionalized after synthesis, in certain cases, such as poly(thiophenol), the microparticles are capable of direct covalent linking with a protein without the need for further modification.

Referring again to FIG. 1, the assay device 20 also contains a detection zone 30 within which is immobilized a first capture reagent that is capable of binding to the analyte or to conjugated detection probes. The binding of the analyte results in a detectible indication that the analyte is present and such an indication may be visual or through other means such as various detectors or readers (e.g., fluorescence readers), discussed below. Readers may also be designed to determine the relative amounts of analyte at the detection site, based upon the intensity of the signal at the detection zone.

In some embodiments, the first capture reagent may be a biological capture reagent. Such biological capture reagents are well known in the art and may include, but are not limited to, antigens, haptens, protein A or G, neutravidin, avidin, streptavidin, captavidin, primary or secondary antibodies (e.g., polyclonal, monoclonal, etc.), and complexes thereof. In many cases, it is desired that these biological capture reagents are capable of binding to a specific binding member (e.g., antibody) present on the detection probes.

It may also be desired to utilize various non-biological materials for the capture reagent. For instance, in some embodiments, the reagent may include a polyelectrolyte. The polyelectrolytes may have a net positive charge or a negative charge, or a net charge that is generally neutral. Some suitable examples of polyelectrolytes having a net positive charge include, but are not limited to, polylysine (commercially available from Sigma-Aldrich Chemical Co., Inc. of St. Louis, Mo.), polyethylenimine; epichlorohydrin-functionalized polyamines and/or polyamidoamines, such as poly(dimethylamine-co-epichlorohydrin); polydiallyldimethyl-ammonium chloride; cationic cellulose derivatives, such as cellulose copolymers or cellulose derivatives grafted with a quaternary ammonium water-soluble monomer; and so forth. In one particular embodiment, CelQuat® SC-230M or H-100 (available from National Starch & Chemical, Inc.), which are cellulosic derivatives containing a quaternary ammonium water-soluble monomer, may be utilized. Some suitable examples of polyelectrolytes having a net negative charge include, but are not limited to, polyacrylic acids, such as poly(ethylene-co-methacrylic acid, sodium salt), and so forth. It should also be understood that other polyelectrolytes may also be used. Some of these, such as amphiphilic polyelectrolytes (i.e., having polar and non-polar portions) may have a net charge that is generally neutral. For instance, some examples of suitable amphiphilic polyelectrolytes include, but are not limited to, poly(styryl-b-N-methyl 2-vinyl pyridinium iodide) and poly(styryl-b-acrylic acid), both of which are available from Polymer Source, Inc. of Dorval, Canada.

The first capture reagent serves as a stationary binding site for complexes formed between the analyte and the detection probes. Specifically, analytes, such as antibodies, antigens, etc., typically have two or more binding sites (e.g., epitopes). Upon reaching the detection zone 30, one of these binding sites is occupied by the specific binding member of the probe. However, the free binding site of the analyte may bind to the immobilized capture reagent. Upon being bound to the immobilized capture reagent, the complexed probes form a new ternary sandwich complex.

The detection zone 30 may generally provide any number of distinct detection regions so that a user may better determine the concentration of a particular analyte within a test sample. Each region may contain the same capture reagents, or may contain different capture reagents for capturing multiple analytes. For example, the detection zone 30 may include two or more distinct detection regions (e.g., lines, dots, etc.). The detection regions may be disposed in the form of lines in a direction that is substantially perpendicular to the flow of the test sample through the assay device 20. Likewise, in some embodiments, the detection regions may be disposed in the form of lines in a direction that is substantially parallel to the flow of the test sample through the assay device.

In conventional lateral flow sandwich devices, uncomplexed analyte would compete with the complexed analyte for the capture reagent located at the detection zone, causing a drop off in the indication of the presence of the analyte. In a graphical representation of signal strength versus time, this drop off resembles a hook, hence this phenomenon is known as the “hook effect”. Depositing the test sample directly on the detection zone 30 results in analyte complexing with the capture reagent before contact with the detection probes. This generally results in all or substantially all of the capture sites of the reagent being occupied by analyte. The detection probes subsequently form the new ternary sandwich complex upon their arrival at the detection zone. This sequence results in the virtual elimination of the “hook effect” found in previous assays because the analyte binds to virtually all of the capture reagent, (provided that there is sufficient analyte) and an excess of detection probes ensures that virtually all capture reagent sites contain complexed analyte.

Referring again to FIG. 1, the porous membrane 22 also contains a control zone 32 positioned downstream from the detection zone 30. The control zone 32 generally provides a single distinct region (e.g., line, dot, etc.), although multiple regions are certainly contemplated by the present invention. For instance, in the illustrated embodiment, a single line is utilized. The control zone 32 may be disposed in a direction that is substantially perpendicular to the flow of the buffer and detection probes through the device 20. Likewise, in some embodiments, the zone 32 may be disposed in a direction that is substantially parallel to the flow through the device 20.

Regardless of its configuration, a second capture reagent is immobilized on the porous membrane 22 within the control zone 32. The second capture reagent serves as a stationary binding site for any detection probes and/or analyte/conjugated probe complexes that do not bind to the first capture reagent at the detection zone 30. Because it is desired that the second capture reagent bind to both complexed and uncomplexed detection probes, the second capture reagent is normally different than the first capture reagent. In one embodiment, the second capture reagent is a biological capture reagent (e.g., antigens, haptens, protein A or G, neutravidin, avidin, streptavidin, primary or secondary antibodies (e.g., polyclonal, monoclonal, etc.), and complexes thereof) that is different than the first capture reagent. For example, the first capture reagent may be a monoclonal antibody (e.g., CRP Mab1), while the second capture reagent may be avidin (a highly cationic 66,000-dalton glycoprotein), streptavidin (a nonglycosylated 52,800-dalton protein), neutravidin (a deglysolated avidin derivative), and/or captavidin (a nitrated avidin derivative). In this embodiment, the second capture reagent may bind to biotin, which is biotinylated or contained on detection probes conjugated with a monoclonal antibody different than the monoclonal antibody of the first capture reagent (e.g., CRP Mab2).

In addition, it may also be desired to utilize various non-biological materials for the second capture reagent of the control zone 32. In many instances, such non-biological capture reagents may be particularly desired to better ensure that all of the remaining conjugated detection probes and/or analyte/conjugated probe complex.

Fluorescence detection may be used to detect the presence of analyte in the detection and control zones and generally utilizes wavelength filtering to isolate the emission photons from the excitation photons, and a detector that registers emission photons and produces a recordable output, usually as an electrical signal or a photographic image. There are generally four recognized types of detectors: spectrofluorometers and microplate readers; fluorescence microscopes; fluorescence scanners; and flow cytometers. One suitable fluorescence detector for use with the present invention is a FluoroLog III Spectrofluorometer, which is sold by SPEX Industries, Inc. of Edison, N.J.

If desired, a technique known as “time-resolved fluorescence detection” may also be utilized in the present invention. Time-resolved fluorescence detection is designed to reduce background signals from the emission source or from scattering processes (resulting from scattering of the excitation radiation) by taking advantage of the fluorescence characteristics of certain fluorescent materials, such as lanthanide chelates of europium (Eu (III)) and terbium (Tb (III)). Such chelates may exhibit strongly red-shifted, narrow-band, long-lived emission after excitation of the chelate at substantially shorter wavelengths. Typically, the chelate possesses a strong ultraviolet absorption band due to a chromophore located close to the lanthanide in the molecule. Subsequent to light absorption by the chromophore, the excitation energy may be transferred from the excited chromophore to the lanthanide. This is followed by a fluorescence emission characteristic of the lanthanide. The use of pulsed excitation and time-gated detection, combined with narrow-band emission filters, allows for specific detection of the fluorescence from the lanthanide chelate only, rejecting emission from other species present in the sample that are typically shorter-lived or have shorter wavelength emission.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A lateral flow assay device for detecting the presence or quantity of an analyte residing in a test sample, said lateral flow assay device comprising a porous membrane, said porous membrane being in communication with a conjugate pad and a wicking pad, said porous membrane defining:
a detection zone where said test sample is applied and within which is immobilized a first capture reagent, said first capture reagent being configured to bind to at least a portion of said analyte and analyte-conjugate complexes to generate a detection signal having an intensity;
a control zone located downstream from said detection zone, wherein a second capture reagent is immobilized within said control zone, said second capture reagent being configured to bind to said conjugate or conjugate-analyte complexes;
said conjugate pad located upstream from said detection zone, said conjugate zone having detection probes with specific binding members for the analyte and;
said buffer release zone located upstream of said conjugate pad and providing for buffer addition to said device, said buffer serving to move said detection probes to said detection zone and to said control zone.

2. A lateral flow assay device as defined in claim 1, wherein said conjugated detection probes comprise a substance selected from the group consisting of chromogens, catalysts, luminescent compounds, radioactive compounds, visual labels, liposomes, and combinations thereof.

3. A lateral flow assay device as defined in claim 1, wherein said conjugated detection probes comprise a luminescent compound.

4. A lateral flow assay device as defined in claim 1, wherein said conjugated detection probes comprise a visual label.

5. A lateral flow assay device as defined in claim 1, wherein said specific binding member is selected from the group consisting of antigens, haptens, aptamers, primary or secondary antibodies, biotin, and combinations thereof.

6. A lateral flow assay device as defined in claim 1, wherein said first capture reagent is selected from the group consisting of antigens, haptens, protein A or G, neutravidin, avidin, streptavidin, captavidin, primary or secondary antibodies, and complexes thereof.

7. A lateral flow assay device as defined in claim 1, wherein said second capture reagent is selected from the group consisting of antigens, haptens, protein A or G, neutravidin, avidin, streptavidin, captavidin, primary or secondary antibodies, and complexes thereof.

8. A lateral flow assay device as defined in claim 1, wherein said analyte is a large pathogen selected from the group consisting of Salmonella species, Neisseria meningitides groups, Streptococcus pneumoniae, Candida albicans, Candida tropicalis, aspergillua, haemophilus influenza, HIV, Trichomonas and Plasmodium.

9. A lateral flow assay device as defined in claim 1, wherein said analyte is selected from the group consisting of toxins, organic compounds, proteins, peptides, microorganisms, amino acids, nucleic acids, hormones, steroids, vitamins, drugs, drug intermediaries or byproducts, bacteria, virus particles and metabolites of or antibodies to any of the above substances.

10. A lateral flow assay device as defined in claim 1, wherein said analyte is a small pathogen.

11. A method for detecting the presence or quantity of an analyte residing in a test sample, said method comprising:
i) providing a lateral flow assay device comprising a porous membrane, in liquid communication with a conjugate pad and a wicking pad, said conjugate pad having detection probes conjugated with a specific binding member for the analyte, said porous membrane defining a detection zone in which a first capture reagent is immobilized and a control zone within which a second capture reagent is immobilized, wherein said control zone is located downstream from said detection zone, said conjugate pad is located upstream of said porous membrane and said buffer release zone is upstream of said conjugate pad;
ii) contacting said test sample containing the analyte with the detection zone;
iii) releasing a buffer at said buffer release zone so that said buffer will carry said detection probes to said detection and control zones;
iv) detecting a detection signal.

12. A method as defined in claim 11, wherein said conjugated detection probes comprise a substance selected from the group consisting of chromogens, catalysts, luminescent compounds, radioactive compounds, visual labels, liposomes, and combinations thereof.

13. A method as defined in claim 11, wherein said conjugated detection probes comprise a visual label.

14. A method as defined in claim 11, wherein said specific binding member is selected from the group consisting of antigens, haptens, aptamers, primary or secondary antibodies, biotin, and combinations thereof.

15. A method as defined in claim 11, wherein said first capture reagent is selected from the group consisting of antigens, haptens, protein A or G, neutravidin, avidin, streptavidin, captavidin, primary or secondary antibodies, and complexes thereof.

16. A method as defined in claim 11, wherein said second capture reagent is selected from the group consisting of antigens, haptens, protein A or G, neutravidin, avidin, streptavidin, captavidin, primary or secondary antibodies, and complexes thereof.

17. A method as defined in claim 11, wherein said second capture reagent comprises a polyelectrolyte.

18. A method as defined in claim 11, wherein said analyte is a large pathogen selected from the group consisting of Salmonella species, Neisseria meningitides groups, Streptococcus pneumoniae, Candida albicans, Candida tropicalis, aspergillua, haemophilus influenza, HIV, Trichomonas and Plasmodium.

19. A method as defined in claim 11, wherein said analyte is selected from the group consisting of toxins, organic compounds, proteins, peptides, microorganisms, amino acids, nucleic acids, hormones, steroids, vitamins, drugs, drug intermediaries or byproducts, bacteria, virus particles and metabolites of or antibodies to any of the above substances.

20. A lateral flow assay device for detecting the presence of an analyte residing in a test sample, wherein detection probes, initially located on a conjugate pad, are moved to a pathogen located in a detection zone having a capture reagent.
